(19) United States
(12) Patent Application Publication
(10) Pub. No.: US 2013/0261674 A1
(43) Pub. Date: Oct. 3, 2013

(54) BONE PLATE POSITIONING DEVICE

(71) Applicant: Daniel Duane Fritzinger, Warsaw, IN (US)

(72) Inventor: Daniel Duane Fritzinger, Warsaw, IN (US)

(21) Appl. No.: 13/436,477

(22) Filed: Mar. 30, 2012

(51) Int. Cl. A61B 17/80

(52) U.S. Cl. 606/286

(57) ABSTRACT

A device to maintain temporary engagement among a bone plate and a bone, the device comprising: (a) a first appendage positioned opposite a second appendage, the first and second appendages including an arcuate vertical profile that partially defines an interior region; (b) a platform concurrently coupled to the first and second appendages, the platform also partially defining the interior region, the platform including a handle and a locator projection extending into the interior region, at least one of the first and second appendages is biased with respect to the platform; and, (c) a first finger grip operatively coupled to at least one of the first and the second appendages, the first projection extending away from the interior region.

### FIELD OF THE INVENTION

The present disclosure is directed to devices used to initially position and retain a bone plate with respect to a bone and, more specifically, includes a clip-on device for use with a volar bone plate to retain the bone plate in position with respect to a radius.

### BRIEF DISCUSSION OF RELATED ART

In the context of distal radius fractures, a bone plate (known as a volar plate) is commonly mounted to the radius (also includes radius bone segments resulting from the fracture) in order to ensure the radius is in a proper orientation to promote bone growth at the fracture site(s). It is often desirable for a surgeon when mounting the volar plate to the radius to temporarily hold the volar plate in the appropriate position before retention screws or other fastening means are concurrently mounted to the volar plate and the radius or radius segments.

Ratcheting forceps are utilized to concurrently retain the volar plate and the radius or radius segments. One of the primary problems with ratcheting forceps is that the forceps tend to be bulky and obstruct an unnecessary amount of the surgeon's working area. Moreover, ratcheting forceps are sometimes difficult to apply and loosen during the surgical procedure.

Accordingly, there is a need for an alternative to ratcheting forceps that may be used to temporarily secure a bone plate to a bone.

### INTRODUCTION TO THE INVENTION

The present disclosure is directed to devices used to initially position and retain a bone plate with respect to a bone. More specifically, the disclosure includes embodiments characterized as clip-on devices that partially circumscribe a bone in order to temporarily retain the position of a bone plate with respect to a bone. While the exemplary embodiment is explained with respect to a volar plate, it should be understood that the exemplary embodiment may be used to temporarily retain the position of a bone plate with respect to any number of bones. Thus, the disclosure is by no means limited to radius fractures and volar plates.

It is a first aspect of the present invention to provide a device to maintain temporary engagement among a bone plate and a bone, the device comprising: (a) a first appendage positioned opposite a second appendage, the first and second appendages including an arcuate vertical profile that partially defines an interior region; (b) a platform concurrently coupled to the first and second appendages, the platform also partially defining the interior region, the platform including a handle and a locator projection extending into the interior region, at least one of the first and second appendages is biased with respect to the platform; and, (c) a first finger grip operatively coupled to at least one of the first and the second appendages, the first projection extending away from the interior region.

In a more detailed embodiment of the first aspect, at least one of the first and second appendages is repositionably mounted to the platform. In yet another more detailed embodiment, the first appendage is pivotally mounted to the platform, and the platform includes a pivot pin around which the first appendage pivots. In a further detailed embodiment, the first appendage is pivotally mounted to the platform, and the first appendage includes a pivot pin pivoting within a first cavity of the platform. In still a further detailed embodiment, the device further includes a spring coupled to the platform and at least one of the first and second appendages to bias at least one of the first and second appendages with respect to the platform. In a more detailed embodiment, both the first and second appendages are repositionably mounted to the platform. In a more detailed embodiment, the platform includes a first pivot pin around which at least one of the first and second appendages pivots. In another more detailed embodiment, at least one of the first and second appendages includes a pivot pin that pivots within a cavity of the platform. In yet another more detailed embodiment, the device further includes a spring coupled to the platform and the first and second appendages to bias the first and second appendages with respect to the platform. In still another more detailed embodiment, the device further includes a first spring coupled to the platform and the first appendage to bias the first appendage with respect to the platform, and a second spring coupled to the platform and the second appendage to bias the second appendage with respect to the platform.

In yet another more detailed embodiment of the first aspect, the platform includes at least one K-wire hole extending into the interior region. In still another more detailed embodiment, the handle and locator projection are removably coupled to the platform. In a further detailed embodiment, the platform includes a through hole sized to receive a portion of the locator projection extending therethrough. In still a further detailed embodiment, the through hole of the platform is partially defined by threads, the locator projection includes threads, and the threads of the platform are sized to engage the threads of the locator projection to facilitate vertical motion of the locator projection with respect to the platform. In a more detailed embodiment, the arcuate vertical profile of the first appendage creates a concave side and an opposite convex side, the arcuate vertical profile of the second appendage creates a concave side and an opposite convex side, the concave side of the first appendage faces the concave side of the second appendage, and the concave sides partially define the interior region. In a more detailed embodiment, the device further includes a second finger grip operatively coupled to the second appendage and extending away from the interior region, wherein the first finger grip is operatively coupled to the first appendage. In another more detailed embodiment, the first finger grip includes a first arcuate depression, the second finger grip includes a second arcuate depression, and the first arcuate depression faces away from the second arcuate depression.

In a more detailed embodiment of the first aspect, the first appendage includes two spaced apart arms that are pivotally coupled to the platform, and the second appendage includes two spaced apart arms that are pivotally coupled to the platform. In yet another more detailed embodiment, each of the two spaced apart arms of the first appendage includes a through orifice, each of the two spaced apart arms of the second appendage includes a through orifice, the platform includes a first pivot pin extending through the through orifice of at least one of the two spaced apart arms of the first appendage to pivotally couple the platform to the first appendage, the platform includes a second pivot pin extending through the through orifice of at least one of the two spaced apart arms of the second appendage to pivotally couple the platform to the second appendage, at least a first portion of the platform extends between the two spaced apart arms of the first appendage, and at least a second portion of the platform extends between the two spaced apart arms of the second appendage. In a further detailed embodiment, the first pivot pin and the second pivot pin are removably mounted to the platform, the first pivot pin extends through both of the through orifices of the two spaced apart arms of the first appendage, and the second pivot pin extends through both of the through orifices of the two spaced apart arms of the second appendage.

It is a second aspect of the present invention to provide a device to maintaining temporary engagement with a bone plate and a bone, the device comprising: (a) a first appendage; (b) a second appendage operatively coupled to the first appendage, the first and second appendages being repositionable with respect to one another, the first and second appendages partially defusing a reconfigurable interior region therebetween; and, (c) a locator projection extending vertically in between the first and second appendages.

In a more detailed embodiment of the second aspect, the device further includes a spring operatively coupled to at least one of the first and second appendages to bias the first appendage with respect to the second appendage. In yet another more detailed embodiment, the device further includes a platform concurrently coupled to the first and second appendages, the platform also partially defining the interior region, the platform including the locator projection. In a further detailed embodiment, the locator projection is removably coupled to the platform.

### BRIEF DESCRIPTION OF THE DRAWINGS

FIG. 1 is an elevated perspective view of a first exemplary bone plate positioning device.

FIG. 2 is an elevated perspective view of the exemplary bone plate positioning device of FIG. 1 shown mounted to a bone and bone plate.

FIG. 3 is a bottom view of the exemplary bone positioning device of FIG. 1.

FIG. 4 is a top view of the exemplary bone positioning device of FIG. 1.

FIG. 5 is an exploded view of the exemplary bone positioning device of FIG. 1.

FIG. 6 is an elevated perspective view of an exemplary appendage of the exemplary bone positioning device of FIG. 1.

FIG. 7 is an elevated perspective view of an exemplary platform of the exemplary bone positioning device of FIG. 1.

FIG. 8 is a profile view of the exemplary bone positioning device of FIG. 1.

FIG. 9 is an elevated perspective view of the exemplary bone plate positioning device of FIG. 14 mounted to a bone and bone plate.

FIG. 10 is a bottom view of the exemplary bone plate positioning device of FIG. 14 mounted to a bone plate.

FIG. 11 is a profile view of the handle and locator projection of the exemplary bone positioning device of FIG. 14.

FIG. 12 is an elevated perspective view of the platform of the exemplary bone positioning device of FIG. 14.

FIG. 13 is a cross-sectional view of the exemplary bone positioning device of FIG. 14.

FIG. 14 is an elevated perspective view of a second exemplary bone plate positioning device.

FIG. 15 is a profile view of the exemplary bone plate positioning device of FIG. 20.

FIG. 16 is an elevated perspective view of the exemplary bone plate positioning device of FIG. 20 mounted to a bone and bone plate.

FIG. 17 is a profile view of an appendage of the exemplary bone positioning device of FIG. 20.

FIG. 18 is an elevated perspective view, from the bottom, of the platform of the exemplary bone positioning device of FIG. 20.

FIG. 19 is an elevated perspective view, from the top, of the platform of the exemplary bone positioning device of FIG. 20.

FIG. 20 is an elevated perspective view of a third exemplary bone plate positioning device.

FIG. 21 is an elevated perspective view of a fourth exemplary bone plate positioning device.

FIG. 22 is a profile view of the exemplary bone plate positioning device of FIG. 21.

FIG. 23 is a profile view of the handle and locator projection of the exemplary bone positioning device of FIG. 21.

FIG. 24 is an elevated perspective view of the exemplary bone plate positioning device of FIG. 21 mounted to a bone and bone plate.

### DETAILED DESCRIPTION

The exemplary embodiments of the present invention are described and illustrated below to encompass devices utilized to temporarily retain the position of a bone plate with respect to a bone and associated methods. Of course, it will be apparent to those of ordinary skill in the art that the preferred embodiments discussed below are exemplary in nature and may be reconfigured without departing from the scope and spirit of the present invention. However, for clarity and precision, the exemplary embodiments as discussed below may include optional steps, methods, and features that one of ordinary skill should recognize as not being a requisite to fall within the scope of the present invention.

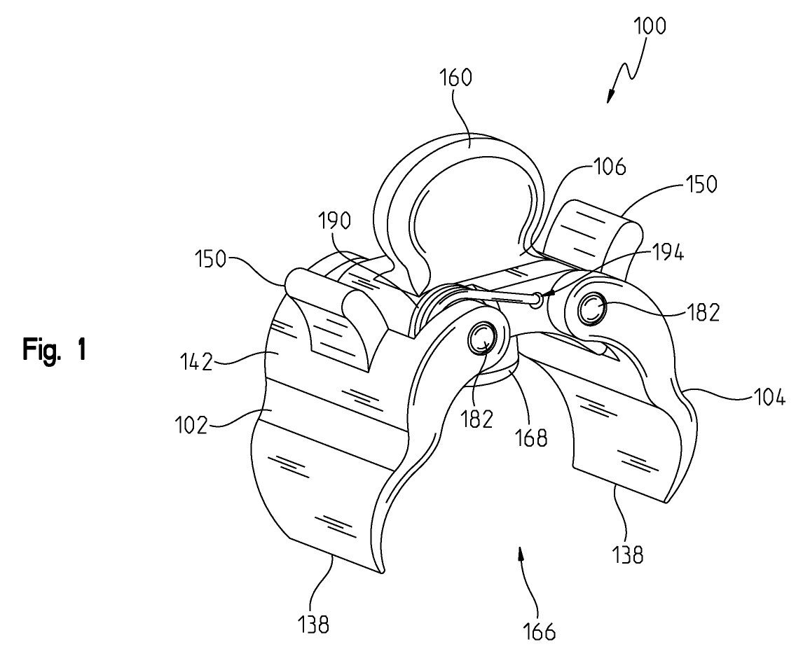

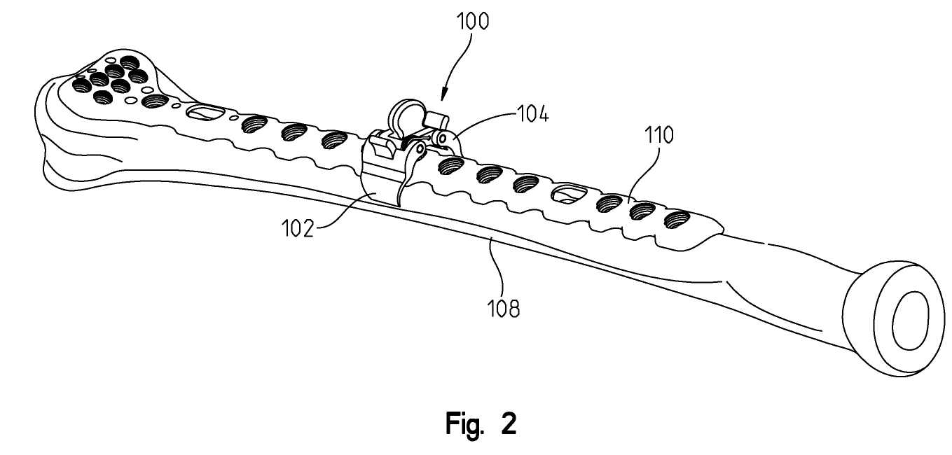

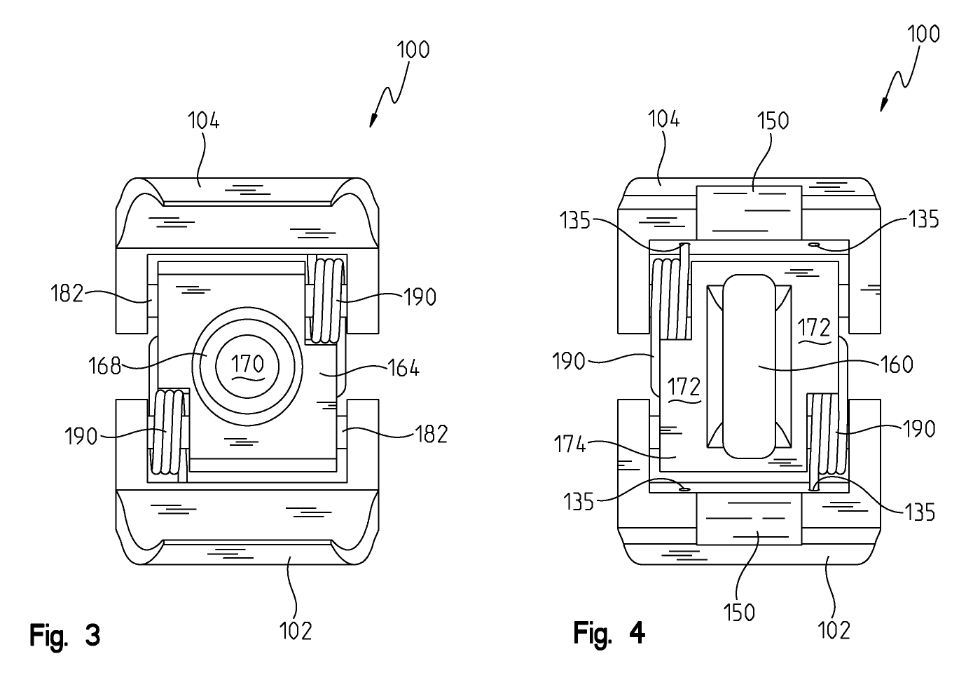

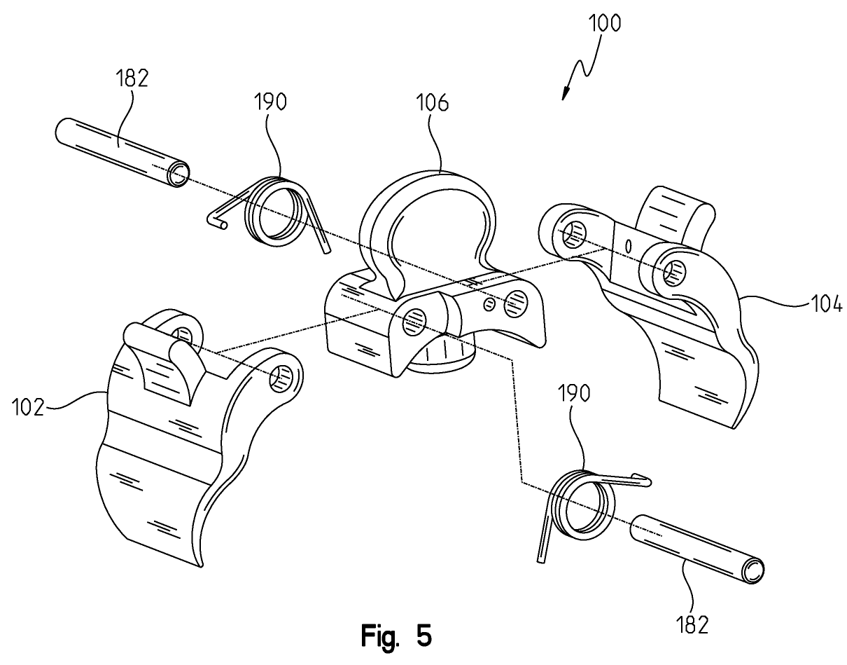

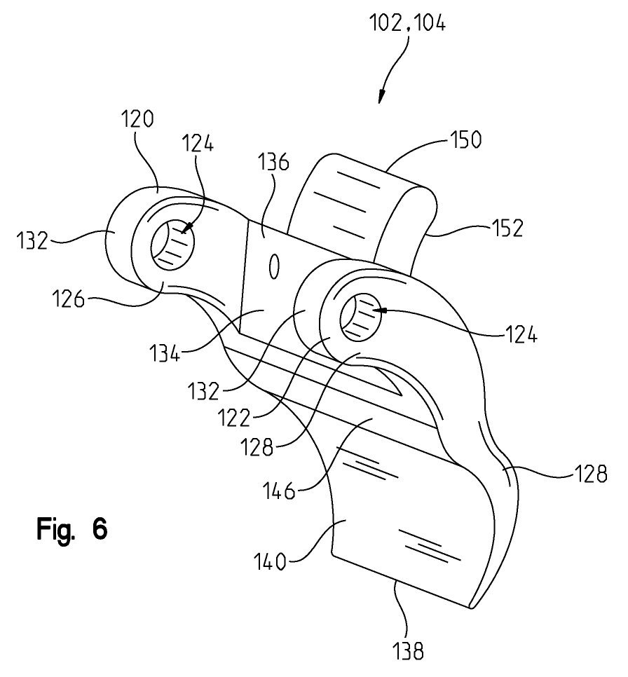

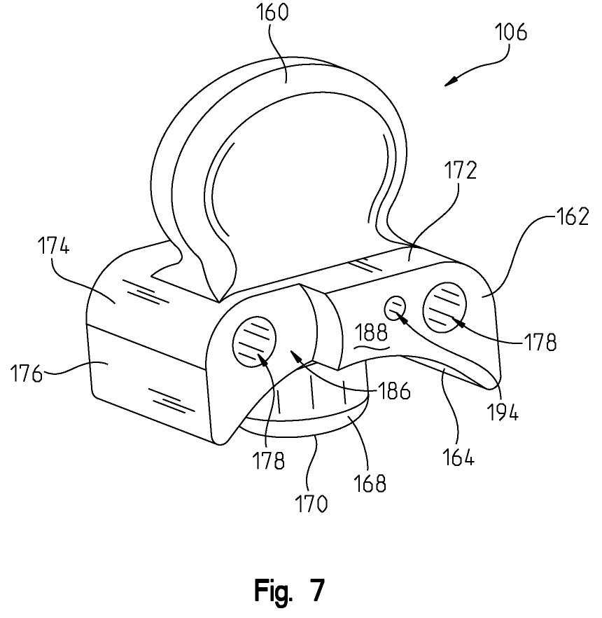

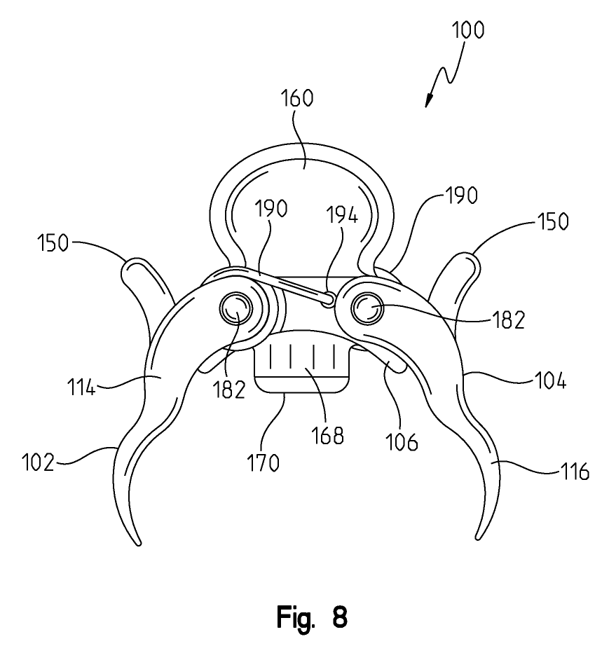

Referencing FIGS. 1-8, a first exemplary device 100 includes a first appendage 102 and a second appendage 104 that are repositionably mounted to a platform 106 to allow the device to partially circumscribe a bone 108 and temporarily mount a bone plate 110 and device 100 to the bone.

Each of the first appendage 102 and the second appendage 104 includes a dual arcuate vertical profile characterized by an upper arcuate profile 114 and a lower arcuate profile 116. The upper arcuate profile 114 helps secure the bone plate 110 in between the appendages 102, 104, while the lower arcuate profile 116 helps secure the bone 108 in between the appendages. In exemplary form, the first and second appendages 102, 104 are identical to one another and oriented opposite one another. For purposes of brevity, only one of the appendages and its features will be discussed in detail.

In this exemplary embodiment, the first appendage 102 includes a pair of spaced apart arms 120, 122 that each includes a through orifice 124 that is circular in cross-section. Each of the arms 120, 122 includes opposing parallel, planar interior and exterior surfaces 126, 128 that are spaced apart from one another by a circumferential surface 132 having a height defined by the thickness of the arm. The arms 120, 122 extend in parallel to one another so that the interior surfaces 126 are parallel to one another and so that the through orifices 124 are axially aligned. The underside of each arm 120, 122, specifically the circumferential surface 132, partially defines the upper arcuate profile 114. Each arm 120, 122 is integrally formed with a vertical wall 136 having a substantially planar interior surface 134 that extends between the respective interior surfaces 126 of the arms and is perpendicular with respect thereto. And each interior surface 134 includes a pair of spring orifices 135.

The exterior surfaces 128 extend vertically to define the side surfaces of the vertical wall 136. But these side surfaces 128 are not parallel to one another as the appendages 102, 104 have a non-uniform width. More specifically, the spacing between the surfaces 128 is at a maximum where the spaced apart arms 120, 122 are located and at a minimum at the tip 138. Interposing the exterior surfaces 128 are inside and outside surfaces 140, 142 that are generally perpendicular with respect to the exterior surfaces and join one another via the circumferential surface 132 and the tip 138. In exemplary form, the contour of the inside and outside surfaces 140, 142 track one another. In other words, portions of the inside surface 140 bow inward and corresponding portions of the outside surface 142 bow outward, while as portions of the inside surface bow outward and corresponding portions of the outside surface bow inward. This is readily apparent when viewing the contour of the inside surface.

Proximate the tip 138 and moving upward, the inside surface 140 bows inward and corresponding portions of the outside surface 142 bow outward, until the inside surfaces changes its curvature at a transition section 146. In the transition section 146, the inside surface 140 changes in curvature to bow outward and the corresponding portion of the outside surface 142 bows inward. More specifically, the transition section 146 separates the upper arcuate profile 114 from the lower arcuate profile 116. While the inside surface 140 is relatively smooth with some undulation, the outside surface is not as smooth.

The outside surface 142 also includes a finger grip 150 that is used to reposition one appendage with respect to the other. In exemplary form, the finger grip 150 comprises an upstanding lever having a concave underside 152 to seat a finger or a portion of a user's hand to actuate the appendage 102, 104. It should be understood that various geometries may be adopted to construct the finger grip 150, with the primary objective of providing a gripping location to actuate one of the appendages 102, 104 with respect to another.

In order to allowing movement of one of the appendages 102, 104 with respect to another, the appendages are mounted to the platform 106. In exemplary form, the platform 106 includes an upstanding handle 160 having a circular profile that extends from a base 162. The base 162 includes its own arcuate profile exhibited by an interior surface 164 that partially defines an interior region 166 in between the appendages 102, 104 and platform 106. The interior surface 164 also has a locator projection 168 extending therefrom. As will be discussed in more detail hereafter, the locator projection 168 is sized to be received within one of a plurality of orifices extending through the bone plate 110 to retard longitudinal motion of the bone plate with respect to the device 100. In exemplary form, the locator projection 168 comprises a cylindrical projection having a substantially flat bottom surface 170.

The top 172 of the base 162 is substantially flat, but for the handle 160, and includes corresponding curved edges 174 that transition into vertical side surfaces 176. Interposing the side surfaces 176 are a pair of through cavities 178 having a longitudinal circular cross-section. Each of the through cavities 178 is sized to accommodate throughput of a cylindrical pivot pin 182. More specifically, the cavity 178 may be slightly smaller than the pivot pin 182 so that the pivot pin is retained in the cavity via a friction fit. Alternatively, or in addition, the pivot pin 182 may be integrally formed or otherwise fastened (i.e., adhesive, welding, etc.) to the base 162 in order to extend longitudinally from opposing ends of the base. In this exemplary embodiment, the pivot pins 182 are cylindrical and sized to allow insertion of the pins into corresponding through orifices 124 of the pair of spaced apart arms 120, 122. In this manner, the appendages 102, 104 are pivotally mounted to the platform 106.

In order to bias the appendages 102, 104 with respect to one another and with respect to the platform 106, the platform includes a cut-out 186 on each longitudinal end 188. The cut-out 186 is sized to accommodate a torsion spring 190 circumscribing one of the pivot pins 182. In this manner, one end of the torsion spring 190 is received within a spring cavity 194 formed into the longitudinal end, while the opposing end of the torsion spring is received within a spring orifice 135 of the interior surface 134 of a corresponding appendage 102, 104.

Materials that may be used to fabricate the foregoing components include any materials that may be used for surgical purposes. In exemplary form, the appendages 102, 104 may be fabricate from metals, ceramics, and polymers including, without limitation, high density polyethylene, titanium, stainless steel, and medical grade ceramics. So too can the platform 106 be fabricated from metals, ceramics, and polymers including, without limitation, high density polyethylene, titanium, stainless steel, and medical grade ceramics. The torsion springs 190 may be fabricated from any metal including, without limitation, titanium, coated steel, and stainless steel. Finally, the pivot pins 182 may be fabricated from metals, ceramics, and polymers including, without limitation, high density polyethylene, titanium, stainless steel, and medical grade ceramics.

When assembled, as shown in FIG. 1, the finger grips 150 for both appendages 102, 104 may be grasped and pulled together so that the tops of the finger grips are moved toward one another to overcome the bias of the torsion springs 190 and increase the distance between the tips 138. The increased distance allows a user to elevate the bone plate into the interior region 166 so that the locator projection 168 is received within one of the through orifices of the bone plate 110. In exemplary form, introduction of the bone plate 110 into the interior region 166 may occur just before or well in advance of the device 100 mounted to the bone 108. In order to mount the device to the bone 108, presuming a portion of the bone plate is already within the interior region 166, the finger grips 150 for both appendages 102, 104 are grasped and pulled together to increase the distance between the tips 138 sufficient to overlap the section of bone in question. Thereafter, the bias of the torsion springs 190 is used to reposition the appendages 102, 104 so that the tips 138 move closer to one another until the appendages contact the bone 108 and form a compression fit to secure the device 100 to the bone. Removal of the device 100 from the bone 108 and bone plate 110 simply involves a reverse process.

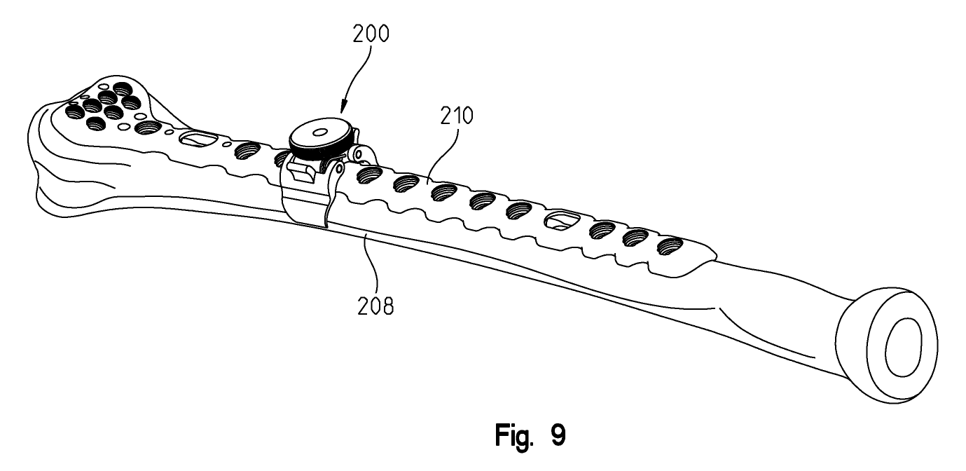

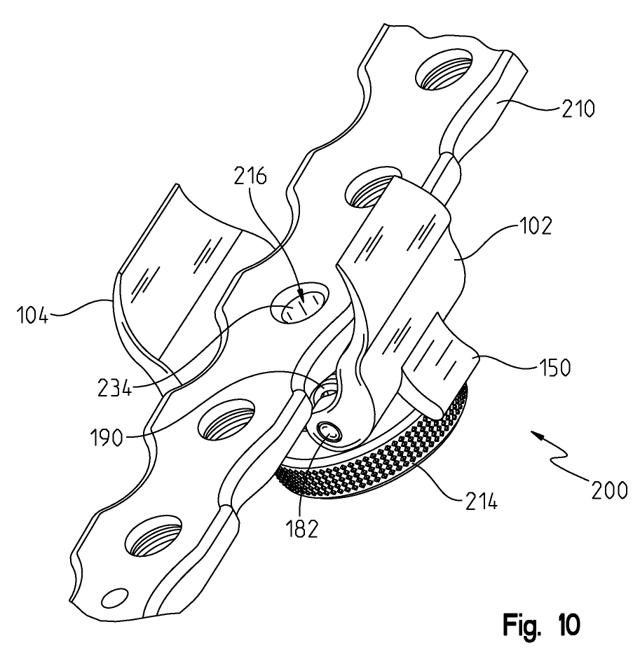

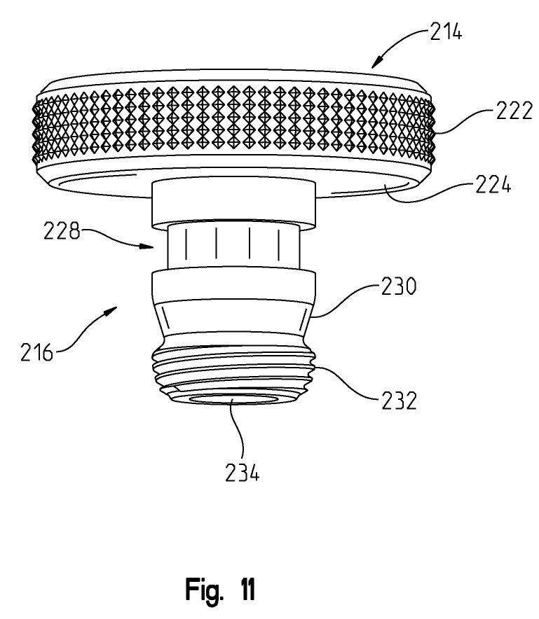

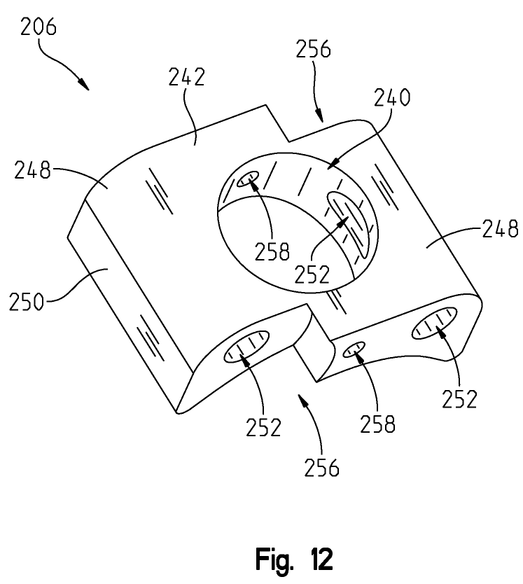

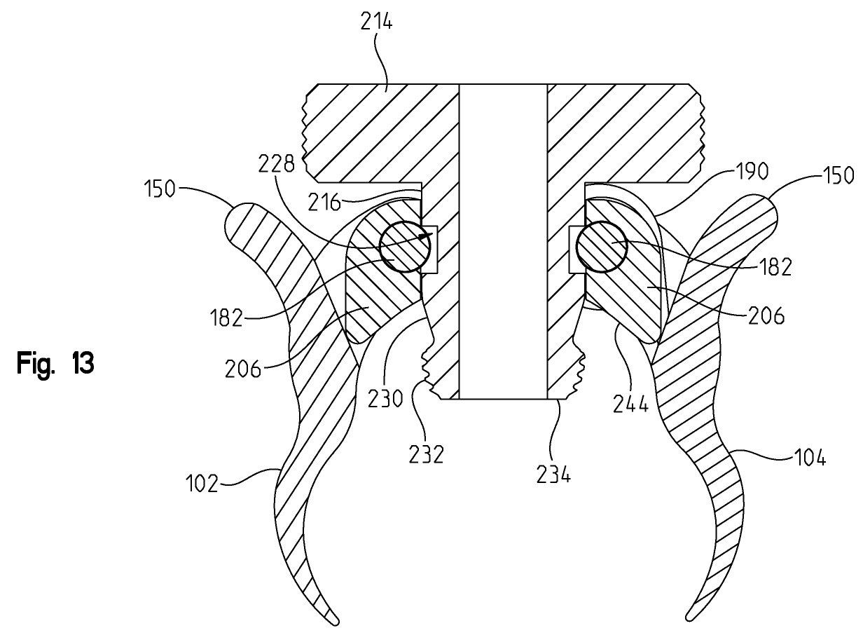

Referring to FIGS. 9-14, a second exemplary device 200 uses the same first and second appendages 102, 104 from the first exemplary device 100. Likewise, the second exemplary device 200 uses the same torsion springs 190 and pivot pins 182 from the first exemplary device 100. Accordingly, a detailed description of the appendages 102, 104, the torsion springs 190, and the pivot pins will not be repeated in furtherance of brevity.

As with the first exemplary device 100, this second exemplary device 200 is repositionably mounted to a platform 206 to allow the device to partially circumscribe a bone 208 and temporarily mount a bone plate 210 and device 200 to the bone. In exemplary form, the platform 206 includes a removable handle 214 that is integrally formed with a locator projection 216. It should be noted, however, that it is within the scope of the invention that the handle 214 and locator projection 216 comprise separate components that are mounted to one another.

In this embodiment, the handle 214 comprises a circular disc having a planar top surface 220 and a circumferential surface 222 having a plurality of bumps to create grip when a user grasps the handle. The circular disc includes a planar bottom surface 224, opposite the top surface 220, that engages the locator projection 216. In exemplary form, the locator projection 216 comprises a cylindrical projection having a circumferential groove 228 that is spaced apart from the bottom surface 224. Adjacent the groove 228 is a frustoconical segment 230 that is coupled to a threaded segment 232 terminating at a tip with a substantially flat bottom surface 234.

The locator projection 216 is adapted to be received within a through opening 240 of the platform 206 that extends from a substantially planar top surface 242 to a bottom arcuate surface 244. The platform 206 includes curved edges 248 that transition into vertical side surfaces 250. Interposing the side surfaces 250 are a pair of through cavities 252 having a longitudinal circular cross-section that also overlap with the through opening 240. Each of the through cavities 252 is sized to accommodate throughput of a cylindrical pivot pin 182. More specifically, the cavity 252 may be slightly smaller than the pivot pin 182 so that the pivot pin is retained in the cavity via a friction fit. Alternatively, or in addition, the pivot pin 182 may be integrally formed or otherwise fastened (i.e., adhesive, welding, etc.) to the platform 206 in order to extend longitudinally from opposing ends of the platform. In this exemplary embodiment, the pivot pins 182 are cylindrical and sized to allow insertion of the pins into corresponding through orifices 124 of the pair of spaced apart arms 120, 122. In this manner, the appendages 102, 104 are pivotally mounted to the platform 206.

In order to bias the appendages 102, 104 with respect to one another and with respect to the platform 206, the platform includes a cut-out 256 on each longitudinal end 188. The cut-out 256 is sized to accommodate a torsion spring 190 circumscribing one of the pivot pins 182. In this manner, one end of the torsion spring 190 is received within a spring cavity 194 formed into the longitudinal end, while the opposing end of the torsion spring is received within a spring orifice 135 of the interior surface 134 of a corresponding appendage 102, 104.

Materials that may be used to fabricate the foregoing components include any materials that may be used for surgical purposes. In exemplary form, the platform 206 may be fabricated from metals, ceramics, and polymers including, without limitation, high density polyethylene, titanium, stainless steel, and medical grade ceramics.

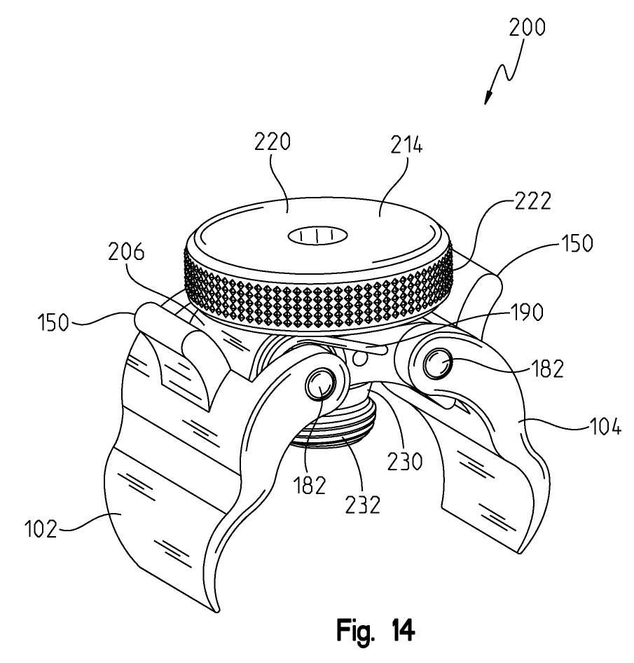

When assembled, as shown in FIG. 14, the pivot pins 182 partially extend into the through opening 240. In this manner, the handle 214 and locator projection 216 may be inserted into the through opening 240 and retained therein by the pivot pins 182 extending into the circumferential groove 228 in the locator projection. In exemplary form, the circumferential groove 228 includes a vertical height that provides for vertical play of the handle 214 and locator projection 216, but resists removal of the locator projection from the through opening 240. Because of the tapered nature of the frustoconical segment 230, at least one of the platform 206, pivot pins 182, and locator projection 216 may be slightly deformed to allow throughput of the locator projection in order to seat the pivot pins within the circumferential groove 228.

In use, the finger grips 150 for both appendages 102, 104 may be grasped and pulled together so that the tops of the finger grips are moved toward one another to overcome the bias of the torsion springs 190 and increase the distance between the tips 138. The increased distance allows a user to elevate the bone plate 210 into the interior region 166 so that the threaded segment 232 of the locator projection 216 is partially received within one of the through orifices of the bone plate 210. Thereafter, rotation of the handle 214 causes the threads of the threaded segment 232 to engage threads on the inside of a corresponding hole of the bone plate 210, thereby drawing the bone plate vertically toward the bottom surface 244 of the platform 206. In exemplary form, introduction of the bone plate 210 into the interior region 166 may occur just before or well in advance of the device 200 mounted to the bone 208.

In order to mount the device 200 to the bone 208, presuming a portion of the bone plate 210 is already within the interior region 166, the finger grips 150 for both appendages 102, 104 are grasped and pulled together to increase the distance between the tips 138 sufficient to overlap the section of bone in question. Thereafter, the bias of the torsion springs 190 is used to reposition the appendages 102, 104 so that the tips 138 move closer to one another until the appendages contact the bone 208 and form a compression fit to secure the device 200 to the bone. Removal of the device 200 from the bone 208 and bone plate 210 simply involves a reverse process.

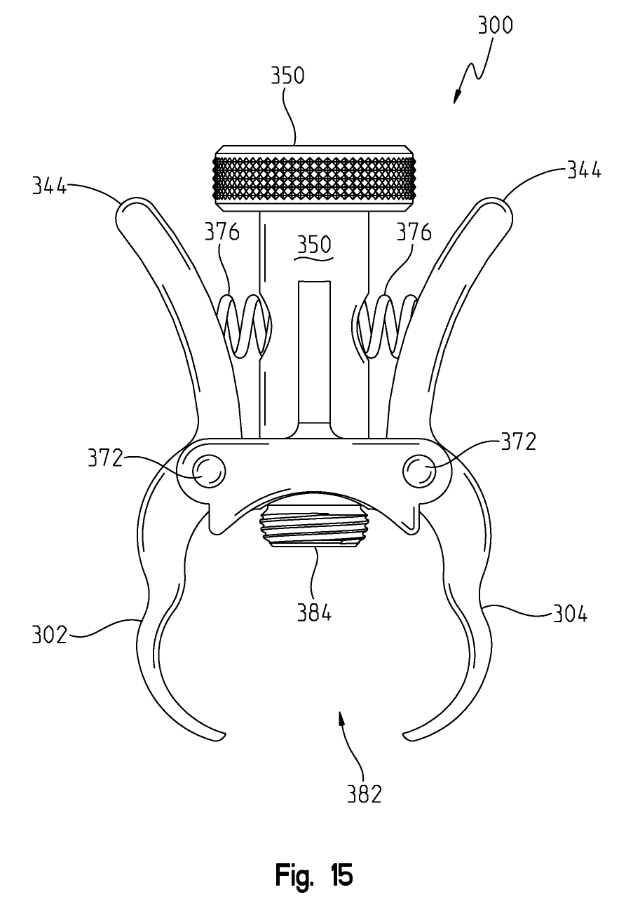

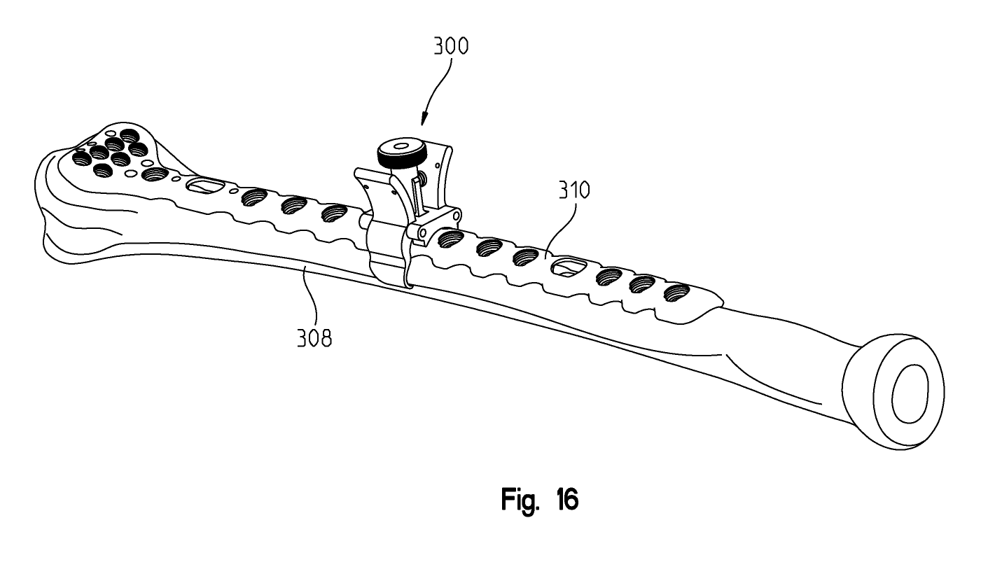

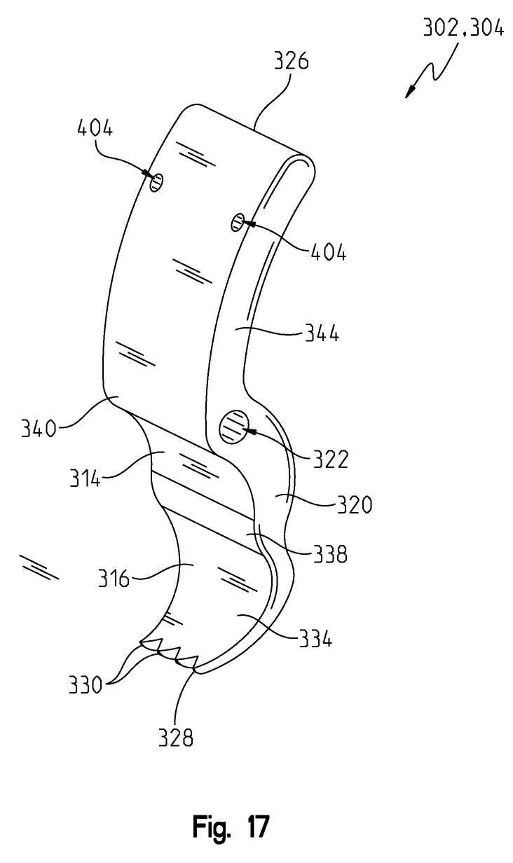

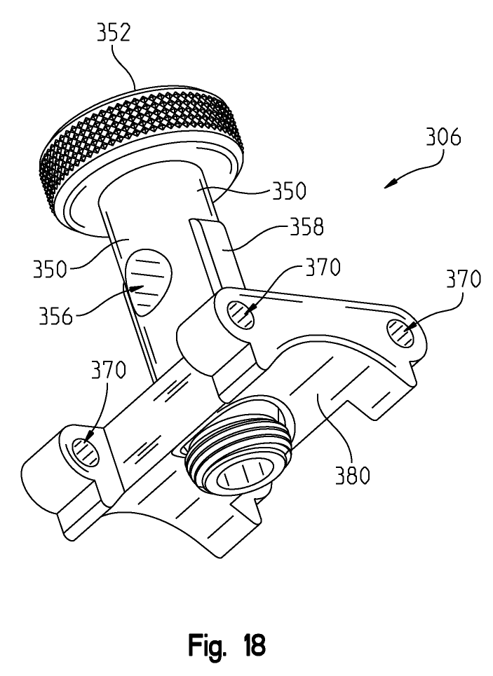

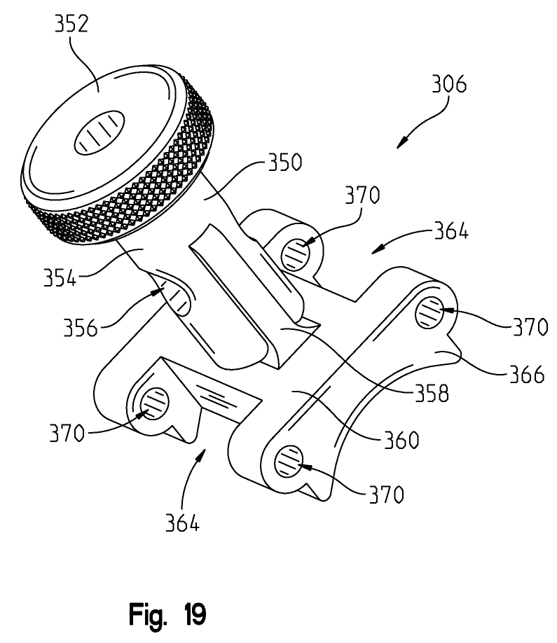

Referencing FIGS. 15-20, a third exemplary device 300 includes a first appendage 302 and a second appendage 304 that are repositionably mounted to a platform 306 to allow the device to partially circumscribe a bone 308 and temporarily mount a bone plate 310 and device 300 to the bone.

Each of the first appendage 302 and the second appendage 304 includes a dual arcuate vertical profile characterized by an upper arcuate profile 314 and a lower arcuate profile 316. The upper arcuate profile 314 helps secure the bone plate 310 in between the appendages 302, 304, while the lower arcuate profile 316 helps secure the bone 308 in between the appendages. In exemplary form, the first and second appendages 302, 304 are identical to one another and oriented opposite one another. For purposes of brevity, only one of the appendages and its features will be discussed in detail.

In this exemplary embodiment, the first appendage 302 includes a pair of exterior side surfaces 320 that extend substantially vertically. But these side surfaces 320 are not parallel to one another as the appendages 302, 304 have a non-uniform width. More specifically, the spacing between the surfaces 320 is at a maximum proximate a longitudinal through orifice 322, where the width is substantially constant up through a proximate tip 326. Extending distally, away from the proximal tip 326, from the through orifice 322 the width of the appendages 302, 304 decreases to a minimum at a distal tip 328. In this exemplary embodiment, the distal tip 328 includes a plurality of teeth 330 to facilitate gripping of the device 300 in tissue underlying the bone 308. Interposing the exterior surfaces 320 are inside and outside surfaces 334, 336 that are generally perpendicular with respect to the exterior surfaces and joint one another at the tips 326, 328. In exemplary from, the contour of the inside and outside surfaces 334, 336 track one another. In other words, portions of the inside surface 334 bows inward and corresponding portions of the outside surface 336 bows outward, while as portions of the inside surface bows outward and corresponding portions of the outside surface bows inward. This is readily apparent when viewing the contour of the inside surface.

Proximate the distal tip 328 and moving proximally, the inside surface 334 bows inward and corresponding portions of the outside surface 336 bow outward until the inside surfaces changes its curvature at a first transition section 338. In the first transition section 338, the inside surface 334 changes in curvature to bow outward and the corresponding portion of the outside surface 336 bows inward. More specifically, the transition section 338 separates the upper arcuate profile 314 from the lower arcuate profile 316. Extending distally from the upper arcuate profile 314, the inside surface 334 bows outward and corresponding portions of the outside surface 336 bow inward until the inside surfaces changes its curvature at a second transition section 340. At this second transition 340, the inside surface 334 bows inward more gradually than is exhibited in the upper and lower arcuate profiles 314, 316 to create an arcuate finger grip 344. At the same time, the outside surface 336 bows outward more gradually than is exhibited in the upper and lower arcuate profiles 314, 316 so that the arcuate profiles of the outside and inside surfaces 334, 336 are substantially the same for the finger grip 344.

In order to allow movement of one appendage with respect to another, the appendages 302, 304 are mounted to the platform 306. In exemplary form, the platform 306 includes an upstanding handle 350 having a circular disc top 352 and a cylindrical shaft 354 extending from the disc top. The shaft includes a cylindrical through opening 356 and a pair of lateral supports 358 is coupled to the shaft 354 and extends longitudinally along the shaft. Both the shaft 354 and lateral supports 358 extend from a top planar surface 360 of the platform 306. Opposing sides of the platform 360 include cut-outs 364 that accommodate portions of the appendages 302, 204. Perpendicular from the cut-outs 364, the longitudinal side surfaces 366 each include a pair of longitudinal through cavities 370 sized to accommodate throughput of a cylindrical pivot pin 372. More specifically, the cavity 370 may be slightly smaller than the pivot pin 372 so that the pivot pin is retained in the cavity via a friction fit. Alternatively, or in addition, the pivot pin 372 may be integrally formed or otherwise fastened (i.e., adhesive, welding, etc.) to the platform 306. In this exemplary embodiment, the pivot pins 372 are cylindrical and sized to allow insertion of the pins into corresponding through orifices 322 of the pair of appendages 302, 304. In this manner, the appendages 302, 304 are pivotally mounted to the platform 306.

In order to bias the appendages 302, 304 with respect to one another and with respect to the platform 306, a compression spring 376 extends through the opening 356 in the shaft 354 to contact opposed inside surfaces 334. As will be discussed in more detail hereafter, the spring 376 may be compressed by movement of the finger grips 344 toward one another, thereby increase a distance between the distal tips 328.

On the opposite side of the compression spring 376, the underside 380 of the platform is arcuately shaped to partially define an interior region 382. Extending distally from the underside 380 is a locator projection 384 co-axial with the shaft 354 that is sized to fit within a hole in the bone plate 310. In this way, the locator projection 384 operates to limit longitudinal travel of the bone plate 310 with respect to the device 300.

Materials that may be used to fabricate the foregoing components include any materials that may be used for surgical purposes. In exemplary form, the appendages 302, 304 may be fabricate from metals, ceramics, and polymers including, without limitation, high density polyethylene, titanium, stainless steel, and medical grade ceramics. So too can the platform 306 be fabricated from metals, ceramics, and polymers including, without limitation, high density polyethylene, titanium, stainless steel, and medical grade ceramics. The compression spring 376 may be fabricated from any metal including, without limitation, titanium, coated steel, and stainless steel. Finally, the pivot pins 372 may be fabricated from metals, ceramics, and polymers including, without limitation, high density polyethylene, titanium, stainless steel, and medical grade ceramics.

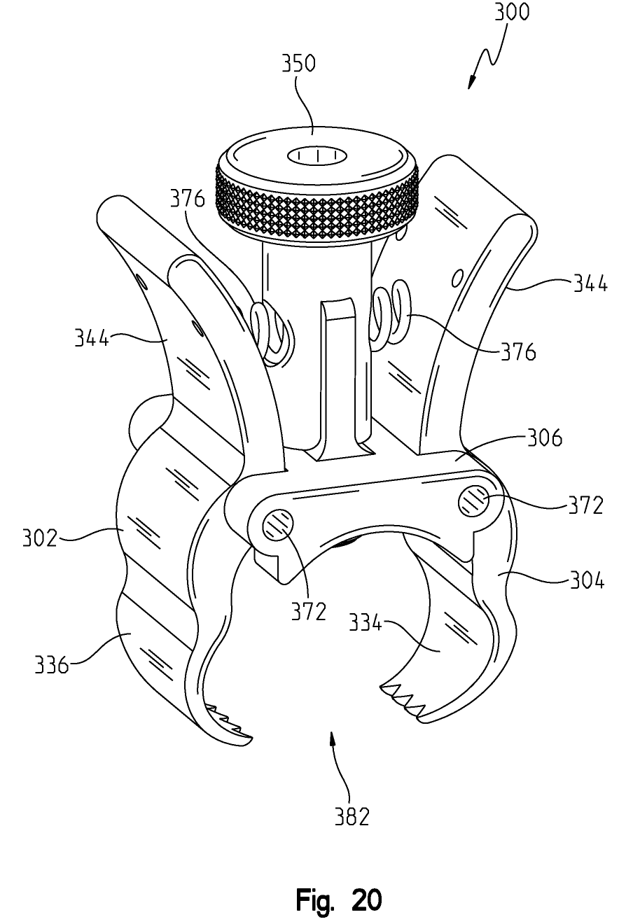

When assembled, as shown in FIG. 20, the finger grips 344 for both appendages 302, 304 may be grasped and pulled together so that the proximal top of each finger grip is moved toward the other to overcome the bias of the compression spring 376 and increase the distance between the tips 328. The increased distance allows a user to elevate the bone plate 310 into the interior region 382 so that the locator projection 384 is received within one of the through orifices of the bone plate 310. In exemplary form, introduction of the bone plate 310 into the interior region 382 may occur just before or well in advance of the device 300 mounted to the bone 308. In order to mount the device 300 to the bone 308, presuming a portion of the bone plate 310 is already within the interior region 382, the finger grips 344 for both appendages 302, 304 are grasped and pulled together to increase the distance between the tips 328 sufficient to overlap the section of bone in question. Thereafter, the bias of the compression spring 376 is used to reposition the appendages 302, 304 so that the tips 328 move closer to one another until the appendages contact the bone 308 and form a compression fit to secure the device 300 to the bone. Removal of the device 300 from the bone 308 and bone plate 310 simply involves a reverse process.

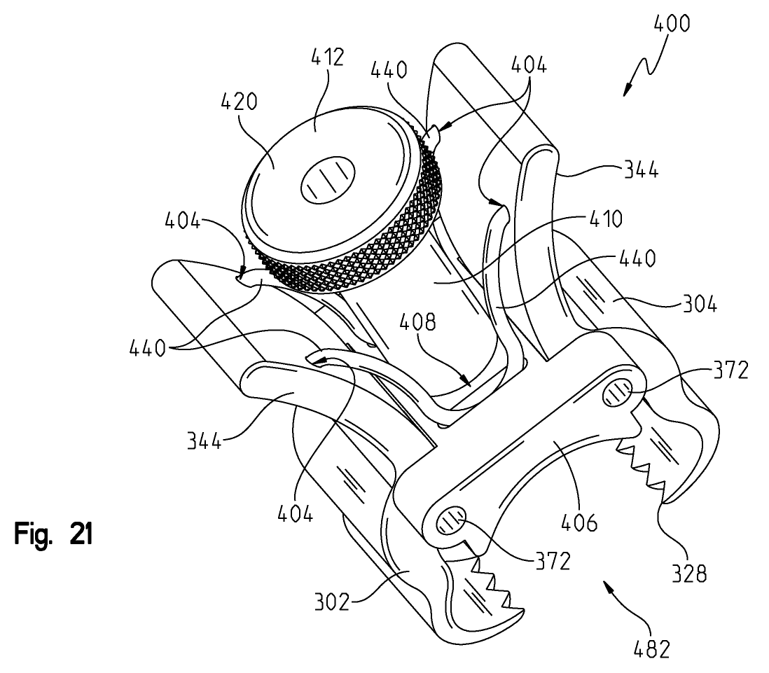

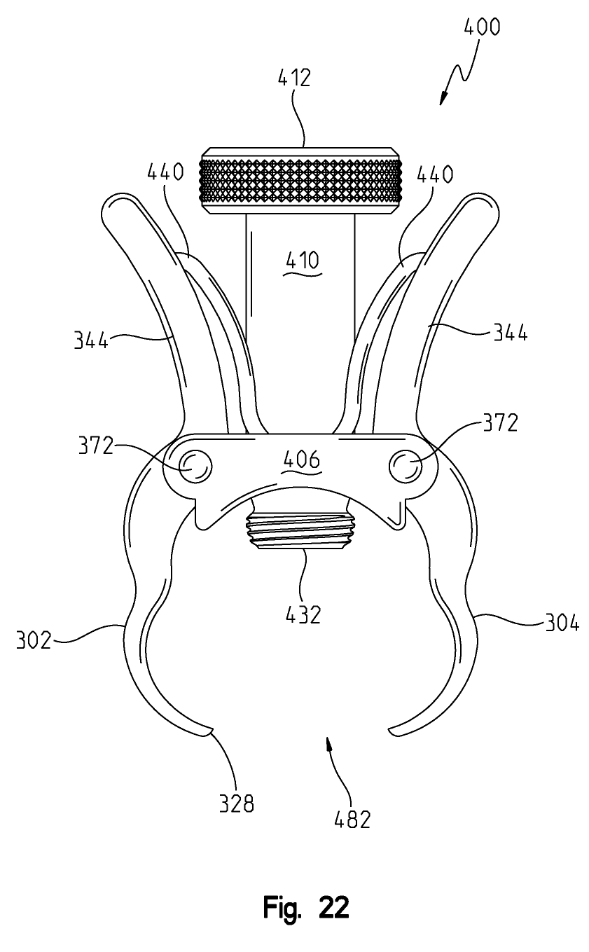

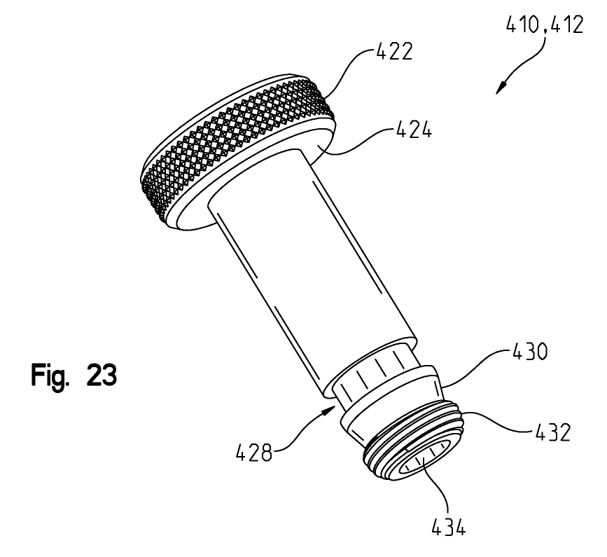

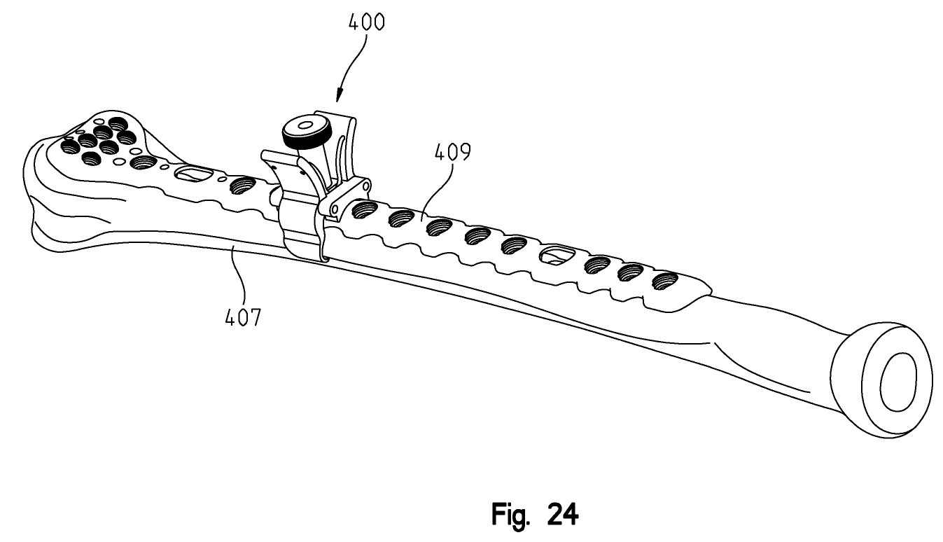

Referring to FIGS. 21-24, a fourth exemplary device 400 uses substantially same first and second appendages 302, 304 from the third exemplary device 100. But in this exemplary embodiment, the first and second appendages 302, 304 each include corresponding orifices 404 to receive an end of a leaf spring 440. Likewise, this fourth exemplary device used the same pivot pins 372. Accordingly, a detailed description of the appendages 302, 304 and pivot pins 372 will not be repeated in furtherance of brevity.

As with the third exemplary device 300, this fourth exemplary device 400 is adapted to partially circumscribe a bone 408 and temporarily mount a bone plate 410 and device 400 to the bone. As with the third exemplary device 300, this fourth exemplary device 400 includes a platform 406 very similar to the platform 306 previously discussed. Unlike the previous platform 306, the instant platform 406 includes a through orifice 408 that accommodates a removable locator projection 410 operatively coupled to a handle 412.

The handle 412 comprises a circular disc having a planar top surface 420 and a circumferential surface 422 having a plurality of bumps to create grip when a user grasps the handle. The circular disc includes a planar bottom surface 424, opposite the top surface 420, that engages the locator projection 410. In exemplary form, the locator projection 410 comprises a cylindrical projection having a circumferential groove 428 that is spaced apart from the bottom surface 424. Adjacent the groove 428 is a frustoconical segment 430 that is coupled to a threaded segment 432 terminating at a tip with a substantially flat bottom surface 434. As discussed above, the locator projection 410 is adapted to be received within the through opening 408 of the platform 406.

In order to bias the appendages 302, 304 with respect to one another and with respect to the platform 406, a pair of leaf springs 440 is mounted to the appendages via the ends of the leaf springs being inserted into the orifices 404. Each leaf spring 440 is outset from the through opening 408.

Materials that may be used to fabricate the foregoing components include any materials that may be used for surgical purposes. In exemplary form, the platform 406 may be fabricated from metals, ceramics, and polymers including, without limitation, high density polyethylene, titanium, stainless steel, and medical grade ceramics. Likewise, the leaf springs 440 may be fabricated from a metal or metal alloy.

When assembled, as shown in FIG. 21, the finger grips 344 for both appendages 302, 304 may be grasped and pulled together so that the proximal top of each finger grip is moved toward the other to overcome the bias of the leaf springs 440 and increase the distance between the tips 328. The increased distance allows a user to elevate the bone plate 410 into the interior region 482 so that the locator projection 410 is received within one of the through orifices of the bone plate 410, presuming the locator projection 410 is received within the through opening 408 of the platform 406. Thereafter, the handle 412 is rotated so that the locator projection 410 is correspondingly rotated in order for the threads of the locator projection to engage the threads of a hole in the bone plate. Continued rotation of the handle 412 is operative to draw the bone plate 410 closer to the underside of the platform 406. In exemplary form, introduction of the bone plate 410 into the interior region 482 may occur just before or well in advance of the device 400 mounted to the bone 408.

In order to mount the device 400 to the bone 408, presuming a portion of the bone plate 410 is already within the interior region 482, the finger grips 344 for both appendages 302, 304 are grasped and pulled together to increase the distance between the tips 328 sufficient to overlap the section of bone in question. Thereafter, the bias of the leaf springs 440 is used to reposition the appendages 302, 304 so that the tips 328 move closer to one another until the appendages contact the bone 408 and form a compression fit to secure the device 400 to the bone. Removal of the device 400 from the bone 408 and bone plate 410 simply involves a reverse process.

Though not specifically discussed, it should be noted that at any of the platforms 106, 206, 306, 406 may include one or more K-wire orifices to accommodate one or more K-wires to align the device 100, 200, 300, 400 with respect to a bone. An exemplary circumstance would be initially positioning one or more K-wires into the bone in question and thereafter sliding the K-wire(s) through respective orifices of the device platform 106, 206, 306, 406 in order to align the device with respect to the bone. Those skilled in the art would be familiar with such a technique in light of the embodiments disclosed herein.

It should also be understood that the dimensions of the device components may be changed to accommodate various sizes and shapes of bones and bone plates. For example, the clip and device may be enlarged for use as a femoral fracture device.

Following from the above description and invention summaries, it should be apparent to those of ordinary skill in the art that, while the methods and apparatuses herein described constitute exemplary embodiments of the present invention, the invention contained herein is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. A device to maintain temporary engagement among a bone plate and a bone, the device comprising:
a first appendage positioned opposite a second appendage, the first and second appendages including an arcuate vertical profile that partially defines an interior region;
a platform concurrently coupled to the first and second appendages, the platform also partially defining the interior region, the platform including a handle and a locator projection extending into the interior region, at least one of the first and second appendages is biased with respect to the platform; and,
a first finger grip operatively coupled to at least one of the first and the second appendages, the first projection extending away from the interior region.

2. The device of claim 1, wherein at least one of the first and second appendages is repositionably mounted to the platform.

3. The device of claim 2, wherein:
the first appendage is pivotally mounted to the platform; and,
the platform includes a pivot pin around which the first appendage pivots.

4. The device of claim 2, wherein:
the first appendage is pivotally mounted to the platform; and,
the first appendage includes a pivot pin pivoting within a first cavity of the platform.

5. The device of claim 2, further comprising a spring coupled to the platform and at least one of the first and second appendages to bias at least one of the first and second appendages with respect to the platform.

6. The device of claim 1, wherein both the first and second appendages are repositionably mounted to the platform.

7. The device of claim 6, wherein the platform includes a first pivot pin around which at least one of the first and second appendages pivots.

8. The device of claim 6, wherein at least one of the first and second appendages includes a pivot pin that pivots within a cavity of the platform.

9. The device of claim 6, further comprising a spring coupled to the platform and the first and second appendages to bias the first and second appendages with respect to the platform.

10. The device of claim 6, further comprising:
a first spring coupled to the platform and the first appendage to bias the first appendage with respect to the platform; and,
a second spring coupled to the platform and the second appendage to bias the second appendage with respect to the platform.

11. The device of claim 1, wherein the platform includes at least one K-wire hole extending into the interior region.

12. The device of claim 1, wherein the handle and locator projection are removably coupled to the platform.

13. The device of claim 12, wherein the platform includes a through hole sized to receive a portion of the locator projection extending therethrough.

14. The device of claim 13, wherein:
the through hole of the platform is partially defined by threads;
the locator projection includes threads; and,
the threads of the platform are sized to engage the threads of the locator projection to facilitate vertical motion of the locator projection with respect to the platform.

15. The device of claim 1, wherein:
the arcuate vertical profile of the first appendage creates a concave side and an opposite convex side;
the arcuate vertical profile of the second appendage creates a concave side and an opposite convex side;
the concave side of the first appendage faces the concave side of the second appendage; and,
the concave sides partially define the interior region.

16. The device of claim 1, further comprising a second finger grip operatively coupled to the second appendage and extending away from the interior region, wherein the first finger grip is operatively coupled to the first appendage.

17. The device of claim 16, wherein:
the first finger grip includes a first arcuate depression;
the second finger grip includes a second arcuate depression; and,
the first arcuate depression faces away from the second arcuate depression.

18. The device of claim 1, wherein:
the first appendage includes two spaced apart arms that are pivotally coupled to the platform; and,
the second appendage includes two spaced apart arms that are pivotally coupled to the platform.

19. The device of claim 1, wherein:
each of the two spaced apart arms of the first appendage includes a through orifice;
each of the two spaced apart arms of the second appendage includes a through orifice;
the platform includes a first pivot pin extending through the through orifice of at least one of the two spaced apart arms of the first appendage to pivotally couple the platform to the first appendage;
the platform includes a second pivot pin extending through the through orifice of at least one of the two spaced apart arms of the second appendage to pivotally couple the platform to the second appendage;
at least a first portion of the platform extends between the two spaced apart arms of the first appendage; and,
at least a second portion of the platform extends between the two spaced apart arms of the second appendage.

20. The device of claim 19, wherein:
the first pivot pin and the second pivot pin are removably mounted to the platform;
the first pivot pin extends through both of the through orifices of the two spaced apart arms of the first appendage; and,
the second pivot pin extends through both of the through orifices of the two spaced apart arms of the second appendage.

21. A device to maintaining temporary engagement with a bone plate and a bone, the device comprising:
a first appendage;
a second appendage operatively coupled to the first appendage, the first and second appendages being repositionable with respect to one another, the first and second appendages partially defining a reconfigurable interior region therebetween; and,
a locator projection extending vertically in between the first and second appendages.

22. The device of claim 21, further comprising a spring operatively coupled to at least one of the first and second appendages to bias the first appendage with respect to the second appendage.

23. The device of claim 22, further comprising a platform concurrently coupled to the first and second appendages, the platform also partially defining the interior region, the platform including the locator projection.

24. The device of claim 23, wherein the locator projection is removably coupled to the platform.
